(19) United States
(12) Patent Application Publication
(10) Pub. No.: US 2015/0038977 A1
(43) Pub. Date: Feb. 5, 2015

(54) Apparatus and method for corneal marking

(71) Applicant: Ravi Nallakrishnan, Westmont, IL (US)

(72) Inventor: Ravi Nallakrishnan, Westmont, IL (US)

(21) Appl. No.: 12/050,167

(22) Filed: Mar. 18, 2008

(51) Int. Cl. A61F 9/013

(52) U.S. Cl. CPC A61F 9/0136; A61B 2019/5437
USPC 606/107

(57) ABSTRACT

An instrument for use in aligning an intraocular lens (IOL) comprises a marker ring rotatably held in a yoke attached to an instrument handle. The ring has an upper surface and a lower surface, and an inner surface and an outer surface. In one version, four marking tabs are attached to the ring outer surface, with each tab spaced apart 90° from its immediate neighbors. Each marking tab has a tab extension that extends below the lower ring surface. The ring also has a pair of axis marking tabs attached to the ring inner surface, with one marking tab positioned diametrically opposite the other. Each axis marking tab has a tab extension that extends above the ring upper surface. Each of the marking tab extensions are coated with a suitable coloring agent and are brought into contact with the cornea, preferably with one marking tab positioned at the limbus, to form reference marks on the cornea. The ring is then rotated in the yoke to bring the axis mounting tabs into position to be brought into contact with the cornea. A keratometer is placed on the cornea and the axis mounting tabs are aligned with markings on the keratometer corresponding to a previously-identified steep axis of the comes. When the axis mounting tabs touch the cornea a pair of axis alignment marks are made which are used as a reference line when the IOL is inserted, allowing the IOL to be properly oriented within the capsular bag.

### BACKGROUND OF THE INVENTION

The present invention relates generally to instruments used in ophthalmic surgery and, more particularly, to instruments used to mark the cornea prior to the implantation and alignment of an intraocular lens (IOL).

Replacement of a cataract with an artificial IOL is now a well-accepted surgical procedure. Typically, during such a procedure the diseased lens is removed from the capsular bag by phacoemulsification and a replacement lens is folded, inserted into the capsular bag and allowed to unfold to act as a replacement lens.

Early implantable IOLs did not afford any correction for corneal astigmatism and a patient suffering from such a condition would still have to wear glasses even after the cataract is removed and a new lens inserted in its place.

Alcon Industries has developed its AcrySof® toric IOL which combines the flexibility of an implantable IOL with the astigmatic corrections available in typical glass or plastic eyeglass lenses. In order to use a toric IOL effectively, the lens must be rotated in the capsular bag to align the lens with a pre-calculated optimal axis, typically the steepest curvature of the cornea. To do so, a keratometer is used to measure the patient's cornea and to determine the steep axis of the cornea. When the toric IOL is implanted, a pair of reference marks on the toric IOL are aligned with the steep axis to provide the desired vision correction.

It is important to have an accurate measurement of the corneal curvature and equally important to find a method for identifying the steep axis during surgery so the IOL can be aligned properly.

The present invention relates to instruments which are used to mark the cornea of the patient to identify pre-phacoemulsification reference points to determine the orientation of the steep axis of the cornea so that after phacoemulsification the IOL can be rotated to align it properly with the steep axis.

To do so, the present invention provides a corneal marker having a marking ring with a front surface and an opposed rear surface. The marking ring is rotatably held in a yoke attached to an instrument handle and the ring is free to rotate 360° within the yoke.

Prior to phacoemulsification the patient's eye is examined and a keratometer is used to determine the angle of the steepest, or “steep” axis along which the astigmatism is most pronounced. The angle is then noted.

A series of four marking tabs are formed on the front surface of the ring placed at 90° degree intervals around the surface of the circular ring. After the marking tabs are coated with dye, one marking tab is aligned with the limbus of the eye and the instrument is then pressed against the cornea to leave marks corresponding to the 3, 6, 9 and 12 numerals on a clock face.

A keratometer is then placed on the eye with the 0° and 180° markings on the keratometer aligned with the marks left on the eye at the corresponding 0° and 180° degree locations. The ring is then rotated with respect to the yoke to allow the rear surface of the ring to come into contact with the cornea. The rear surface has a pair of axis marking tabs at the 0 and 180° positions on the ring.

The axis tabs are then coated with dye and the instrument is then moved to align the second surface with the cornea and to press the axis tabs against the cornea with the marking tabs aligned with the angle marking on the keratometer that corresponds to the steep axis on the cornea. The axis tabs make a pair of marks on the cornea, and it is this second set of reference marks that identifies the axis with which the IOL is aligned when it is inserted so that the stigmatic correction of the IOL is maximized.

The ability of the ring to rotate within the yoke makes it possible for the surgeon to hold the instrument in a variety of hand positions and still place the marking ring against the surface of the cornea. For example, the surgeon may use a limbal, superior or inferior approach to place the instrument on the eye and may adjust the position of the ring to accommodate this preference. It is also possible to use the instrument on both the left and right eye with equal facility.

U.S. Pat. No. 4,739,761 teaches and describes a cornea marker that employs a rotating marker wheel to allow the cornea to be marked at selected locations.

It is an object of the present invention to provide instruments useful for marking the cornea for the insertion and alignment of a multifocal IOL while allowing the surgeon to double check the location of the corneal steep axis prior to insertion of the lens.

It is a further object of the present invention to provide marking rings on such instruments that are rotatably grasped within an instrument handle in order to provide a variety of angles at which the instrument may be held and still manipulated to properly mark the cornea.

### BRIEF DESCRIPTION OF THE DRAWINGS

These and further objects of the present invention will become more apparent upon considering the accompanying drawings in which:

FIG. 1 is a perspective view of a prior art corneal reference marker;

FIG. 2 is a detail of the marking end of the marker shown in FIG. 1;

FIG. 3 is a perspective view of a prior art corneal axis marker;

FIG. 4 is a top detail view of the marking end of the marker in FIG. 3;

FIG. 5 is a bottom detail view of the marker in FIG. 3;

FIG. 6 is a front elevation of an instrument embodying certain of the principles of the present invention;

FIG. 7 is an enlarged detail of the yoke and ring of FIG. 6;

FIG. 8 is a perspective view of the marking ring of FIG. 6;

FIG. 9 is a top plan view of the ring in FIG. 8;

FIG. 10 is a view along 10-10 of FIG. 9;

FIG. 11 is a lateral schematic view showing the marking ring disposed at an angle to the instrument handle;

FIG. 12 is a view of a human eye with reference marks thereon;

FIG. 13 is schematic view of a keratometer positioned on a human eye;

FIG. 14 is a perspective view showing the marking ring (with the handle removed for clarity) disposed within the keratometer;

FIG. 15 is a view of a human eye with the reference and index marks thereon; and

FIG. 16 is a view of a foldable IOL with astigmatism correction.

### DETAILED DESCRIPTION OF THE DRAWINGS

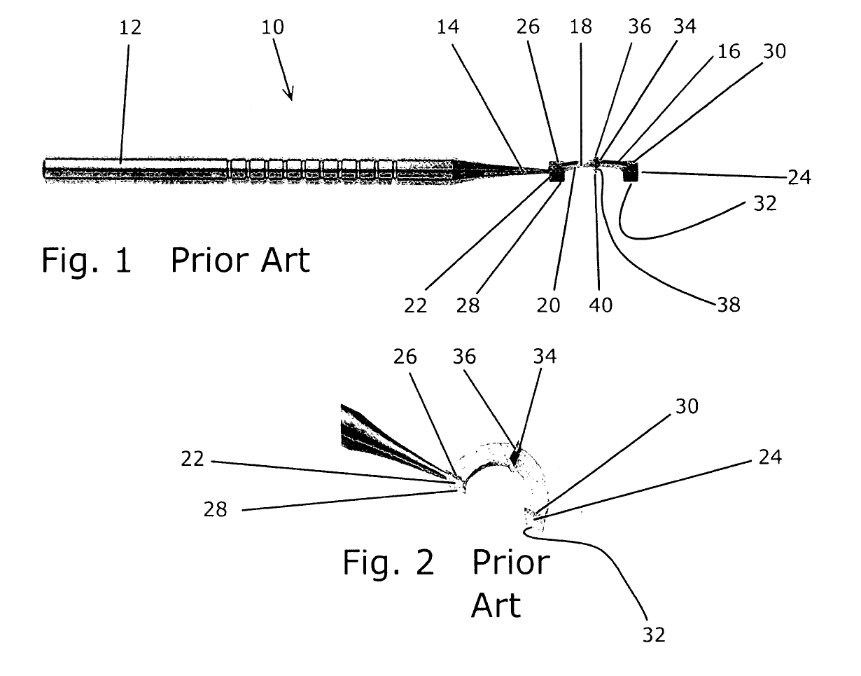

Referring now to FIG. 1, the numeral 10 identifies a prior art reference marker. Reference marker 10 has a handle 12 tapering at one end to form a throat 14 to which a marker blade 16 is integrally, fixedly and non-rotatably attached. As seen in FIG. 1, blade 16 has an upper surface 18 and a lower surface 20 and is preferably formed as a semicircular flat segment. As best seen in FIG. 2, blade 16 has first and second marking tabs 22, 24 formed diametrically opposite one another and formed integrally with blade 18. Tab 22 has an upper marking edge 26 and a lower marking edge 28 while tab 24 has an upper marking edge 30 and a lower marking edge 32.

A third marking tab 34 is formed integral with upper surface 18 and midway along blade 16 between first and second marking tabs 22, 24. Tab 34 has an upper marking edge 36. A fourth marking tab 38 having a lower marking edge 40 extends from lower surface 20 opposite third marking tab 34.

While the marking tabs 22, 24, 34 and 38 are shown in FIGS. 1 and 2 as elongated “knife edges” other shapes can be used for the marking tabs. For example, raised hemispherical dots can also be used. The shape of the marking tab can determine the shape and size of the mark left on the cornea.

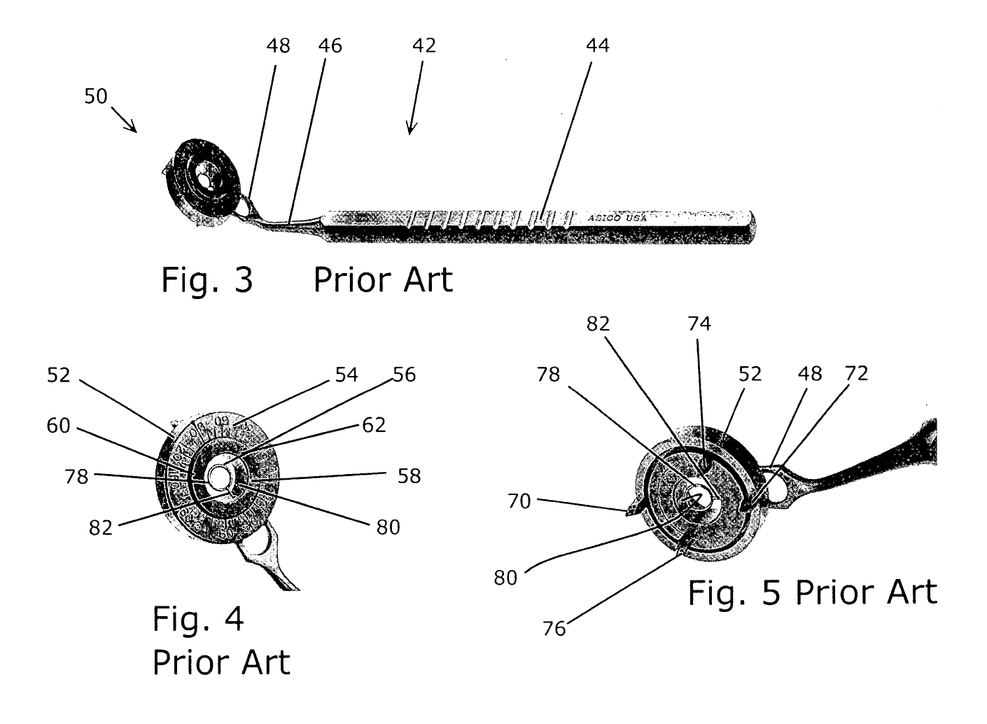

Referring now to FIG. 3, the numeral 42 identifies an axis marker having a handle 44 tapering to a throat portion 46 to which a mounting fork 48 is integrally attached at a preselected and nonadjustable angle.

Attached to fork 48 is a combined gauge and keratometer assembly 50. As best seen in FIGS. 4 and 5, assembly 50 comprises a toroidal gauge ring 52 having an upper surface 54 onto which a scale marked off in degrees from zero to 180 is engraved. Ring 52 is attached to fork 48 such that a 90° marking on the scale is positioned at fork 48. Ring 52 does not rotate with respect to fork 48.

Gauge ring 52 has a central circular aperture 56 formed therethrough. An inner toroidal marker ring 58 is rotatably fitted to gauge ring 52 through aperture 56. Ring 58 has a first right circular segment 60 held rotatably within the gauge ring 52 with first segment 60 extending above upper gauge ring surface 54. A reference mark 62 is engraved on ring 58.

Referring now to FIG. 5, pair of locating tabs 70, 72 are formed on the lower surface of gauge ring 52 preferably to coincide with the 90/90° marks on top surface 54 of ring 52. Also as seen in FIG. 5, a pair of marking tabs 74, 76 are formed on the lowermost surface of third marker ring segment 64. As can be appreciated, marking edges 74, 76 will rotate as marker ring 58 is rotated.

A keratometer ring 78 is attached to inner wall 80 of marker ring 58 by ring shaft 82. When axis marker 42 is placed on a patient's cornea, light from the operating microscope is directed through keratometer ring 78 and will highlight the general shape of any astigmatism in the cornea. This is not intended as a precise identification of the position of the “steep axis” of the cornea, but is intended to provide a backup indicator to confirm to the surgeon that the previously obtained keratometer readings were correct in identifying the steep axis.

In use, marking tabs 74, 76 are coated with a suitable dye and marker ring 58 is rotated to bring reference mark 62 in alignment with the scale scribed on surface 54 to coincide with the angle of the previously-measured steep axis. Non-rotating markers 70, 72 are then coated with a suitable dye. The instrument is then placed on the eye to bring one of the non-rotating tabs 70, 72 at the corner of the eye such that tabs 74, 76 are in alignment with the steep axis. Tabs 74, 76 are then pressed against the cornea to leave a pair of marks that allow the surgeon to align the IOL along the steep axis after insertion.

As shown in FIGS. 4 and 5, ring 78 is formed with a single ring, but multiple concentric rings can also be used to provide differing light patterns and effects as desired.

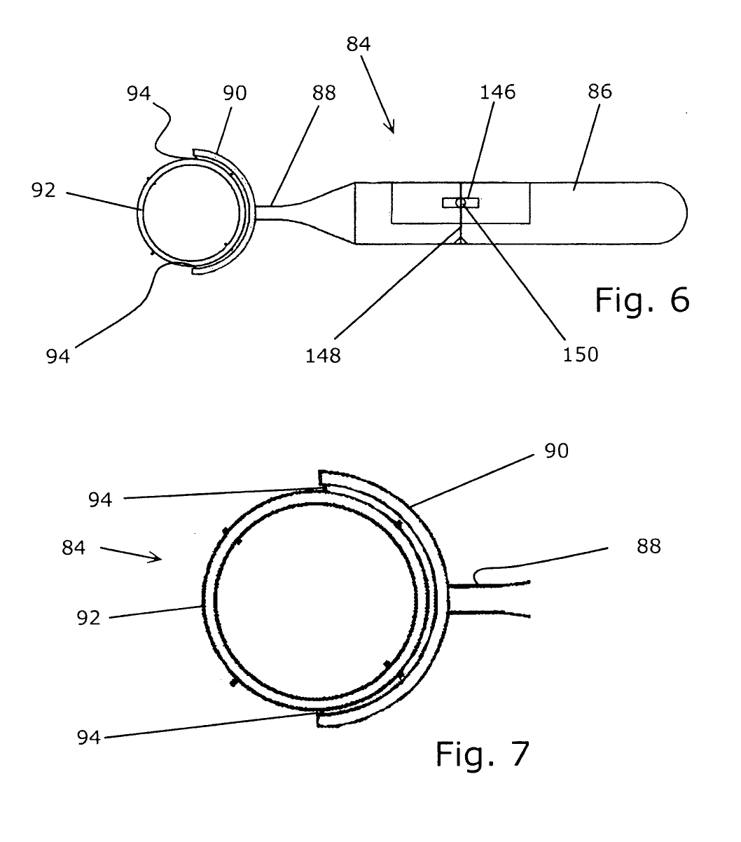

Referring now to FIGS. 6 and 7, the numeral 84 identifies generally a corneal marking instrument having a handle 86 tapering to a throat 88 to which a generally semi-circular yoke 90 is integrally attached. A marking ring 92 is rotatably attached to yoke 90 at pivots 94 and yoke 90 is sized to allow marking ring 92 to be freely rotated throughout at least a 360° range.

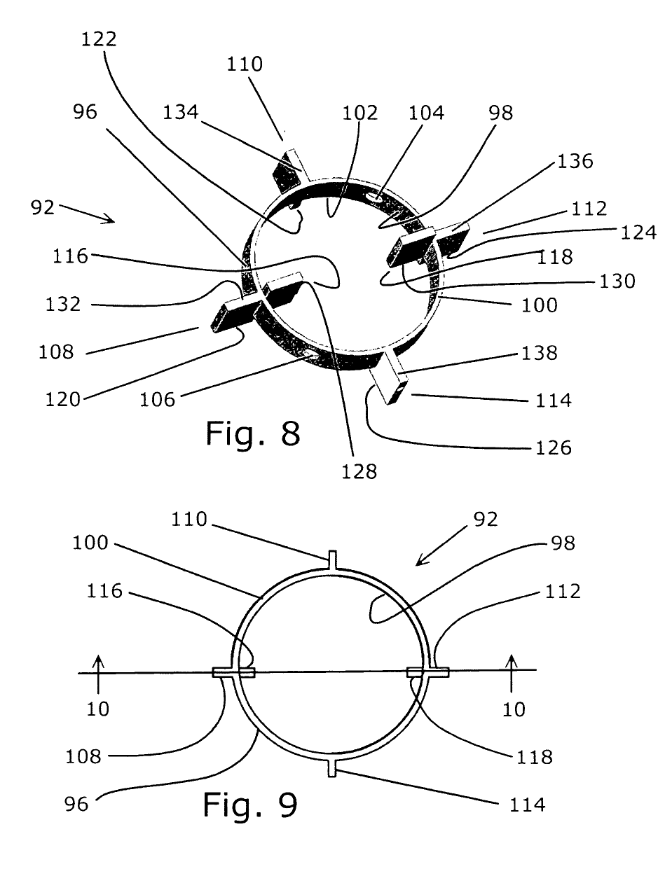

Referring now to FIG. 8, marking ring 92 is shown in perspective as removed from instrument 84. In the embodiment shown, ring 92 is formed as a circular torus having an outer wall surface 96, an inner wall surface 98, an upper surface 100 and a lower surface 102. Mounting ports 104, 106 comprise the attachment points to ring pivots 94.

Referring now to FIGS. 8 and 9, a series of four reference markers 108, 110, 112 and 114 are formed integrally with and about the periphery of ring 92 at outer wall 96. In the embodiment shown, reference markers 108, 110, 112 and 114 are formed at 90 degree intervals. Also shown in FIGS. 8 and 9 are axis markers 116, 118, formed integrally with ring 92 at inner wall 98.

Thus, reference markers 108, 110, 112 and 114 extend outward from outer wall 96 of ring 92 while axis markers 116, 118 extend inward from inner wall 98 of ring 92.

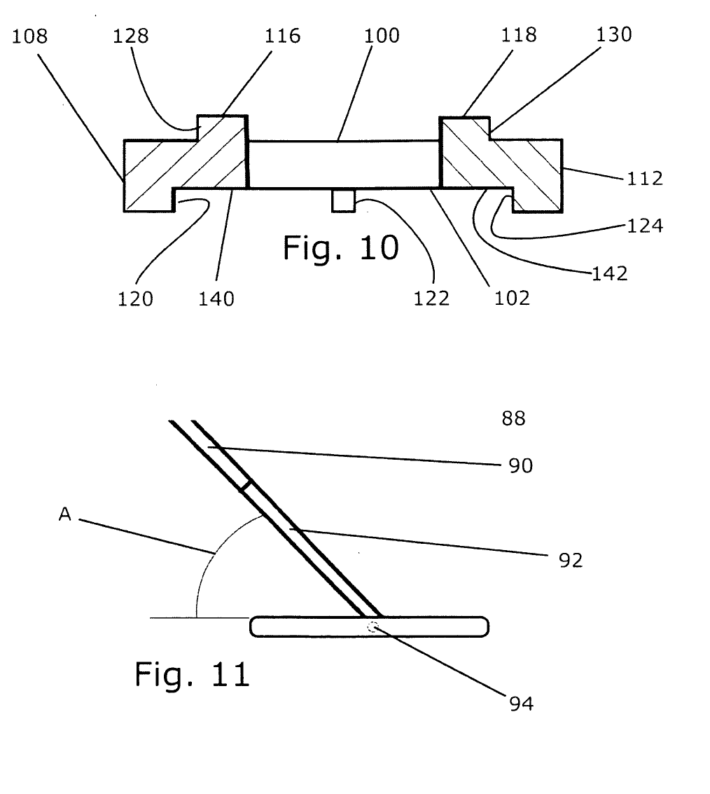

Referring now to FIGS. 8 and 10 it can be seen that reference marker 108 has a marker tab 120 formed integrally therewith and extending below lower surface 102. In like fashion, reference marker 110 has a tab 122 formed integrally therewith and extending below surface 102, reference marker 112 has a tab 124 formed integrally therewith and extending below surface 102 and reference marker 114 has a tab formed integrally therewith a tab 126 formed integrally therewith and extending below lower surface 102.

As seen in FIG. 10, reference marker 116 has an extension tab 128 extending above upper surface 100 and reference marker 118 has an marker tab 130 extending above upper surface 100.

As seen in FIGS. 8 and 10 surfaces 132, 134, 136 and 138 of reference markers 108, 110, 112 and 114, respectively, do not extend above upper surface 100 to a distance greater than that of marker tabs 128, 130. In the embodiment shown, surfaces 132, 134, 136 and 138 are coextensive with upper surface 100. In similar fashion, the lower surfaces 140, 142 of axis markers 116, 188, respectively, do not extend below lower surface 102 to a distance greater than that of marker tabs 120, 122, 124 and 126. In the embodiment shown, lower surfaces 140, 142 are coextensive with lower surface 102.

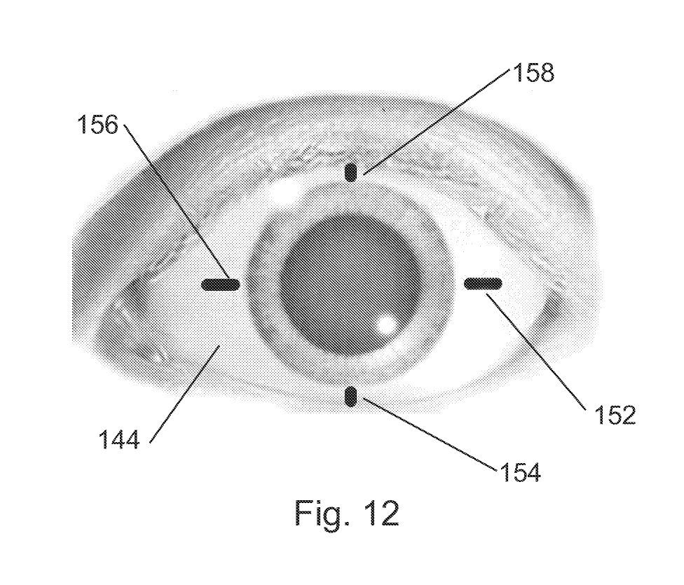

Referring now to FIG. 11, throat 88 and yoke 90 are shown pivoted about pivots 94 to place yoke 90 at an angle A with respect to ring 92. It is a feature of the present invention that throat 88 and yoke 90 may be pivoted completely about ring 92 through an arc of rotation of 360°. This allows the surgeon to set ring 92 at any desired angle with respect to yoke 90 and, thereafter, to place ring 92 on a corneal surface 144 as seen in FIG. 12.

In use, while a patient is seated, marker tabs 120, 122, 124 and 126 of ring 92 are coated with a surgically-acceptable dye, such as gentian blue or gentian violet. Next one of the reference markers is aligned with the limbus of the eye and handle 86 of instrument 84 is held in a horizontal position. To assist in aligning the instrument horizontally a spirit level 146 is positioned in handle 86 and a laser-scribed line 148 is formed on spirit level 146 and handle 148 as a reference line for the bubble 150 at spirit level 146 as seen in FIG. 6.

In a preferred embodiment spirit level 146 may be removed from handle 148 for cleaning and sterilization.

Referring again to FIG. 11, yoke 90 can be positioned at any angle with respect to marking ring 92 to facilitate the surgeon's task in aligning one of the reference markers with the limbus.

Once horizontal alignment has been achieved, reference markers 108, 110, 112 and 114 are pressed against corneal surface 144. As seen in FIG. 12, four reference marks 152, 154, 156 and 158 are formed on the corneal surface 144 at the 3, 6, 9 and 12 clock positions.

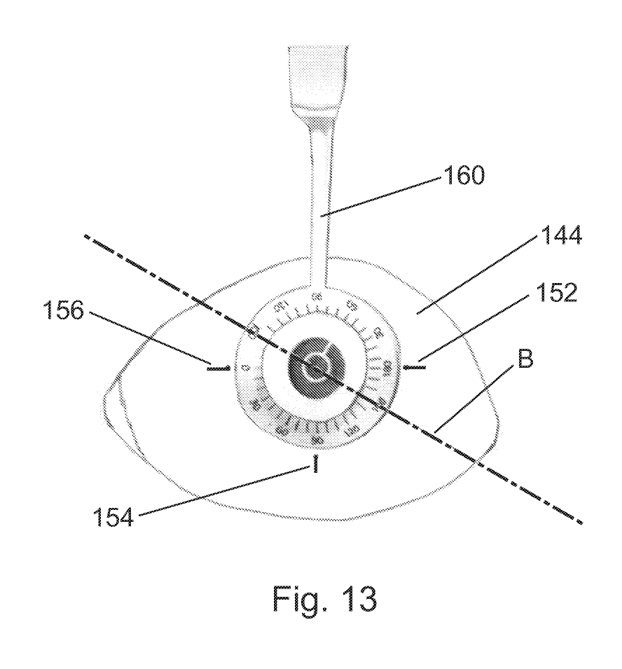

As described above, prior to surgery the patient's steep axis is measured. As seen in FIG. 13 a keratometer 160 is placed on the cornea, preferably aligned with marks 152, 156, and is used to determine the steep axis B along which the IOL to be inserted must be aligned.

To mark the axis or position of the steep axis, axis marker marker tabs 116, 118 are coated with a suitable coloring agent or dye and ring 92 is rotated with respect to yoke 90 to position marker tabs 116, 118 for contact with corneal surface 144.

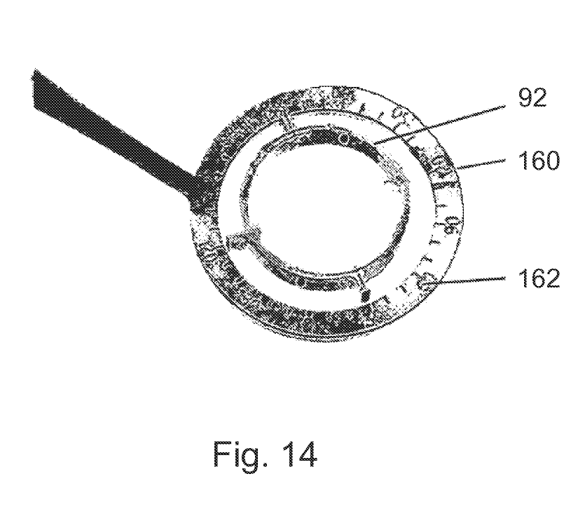

As seen schematically in FIG. 14, ring 92 is shown (with handle 86 removed for clarity) and along with keratometer 160 is positioned on the cornea, centered on the pupil and aligned such that the 0 degree reading on the scale is aligned with reference mark 156. Ring 92 is then aligned with the scale reading on scale 162 that coincides with the steep axis 164 of the cornea such that marker tabs 116, 118 are positioned on the steep axis.

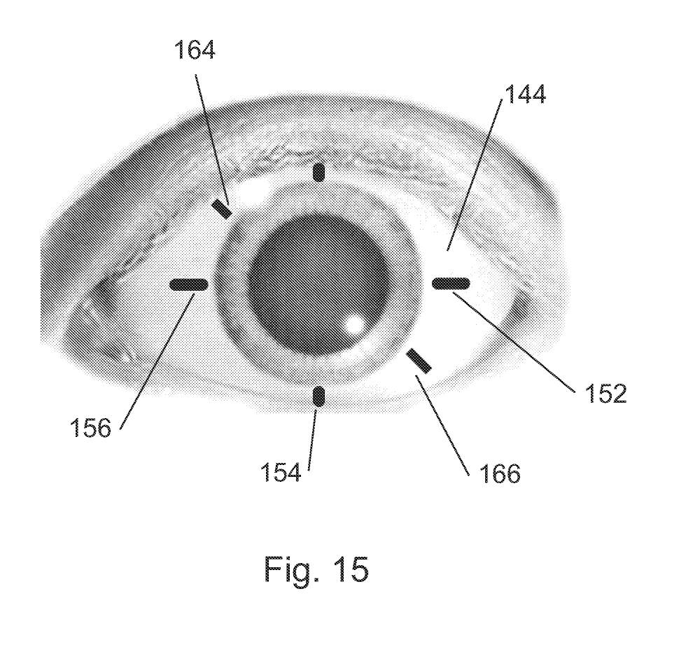

When marker tabs 116, 118 are aligned with the steep axis, ring 92 is pressed against corneal surface 144 and, as seen in FIG. 15, axis reference marks 164, 166 are made on corneal surface 144, defining steep axis B.

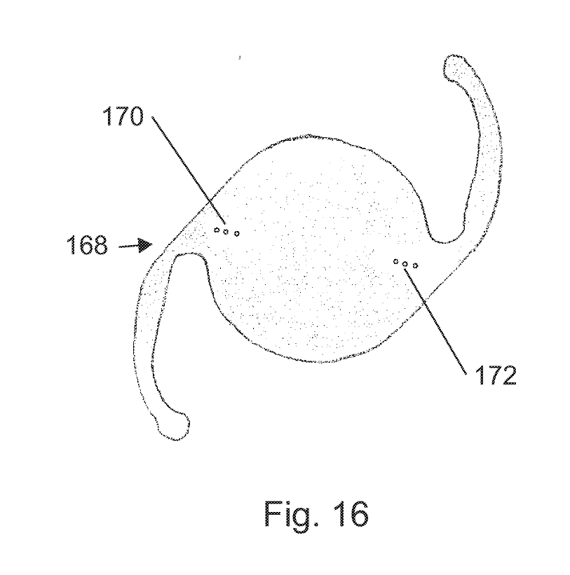

Referring now to FIG. 16, the numeral 168 identifies a plastic IOL such that manufactured under the AcrySof® trademark by Alcon Laboratories, Inc. IOL 168 has a pair of reference mark groupings 170, 172 which, when aligned with steep axis B defined by reference marks 164, 166 provides the astigmatism correction required.

The surgeon can double check the alignment of the IOL by observing if the IOL is lined up with the corneal steep axis as determined by the marks 164, 166.

While the foregoing describes a preferred embodiment or embodiments of the invention, it is to be understood that this description is made by example only and is not intended to limit the scope of the present invention. It is expected that alterations and further modifications, as well as other and further applications of the principles of the present invention will occur to others skilled in the art to which the invention relates, and while differing from the foregoing, remain within the spirit and scope of the invention as herein described and claimed. Where means-plus-function clauses are used in the claims, such language is intended to cover the structures described herein as performing the recited functions, and not only structural equivalents but equivalent structures as well. For the purposes of the present disclosure, two structures that perform the same function within an environment described above may be equivalent structures.

I claim:

1. A marking instrument for marking a patient's cornea, said instrument comprising:
an instrument handle;
a yoke attached at one end of said handle,
said yoke having first and second pivot mounts mounted thereon;
a marking ring,
said marking ring formed as a torus and having an upper surface, a lower surface opposite said upper surface, an outer surface extending between said upper and lower surfaces, and an inner surface opposite said outer surface;
said marking ring having first and second pivot ports formed therethrough extending from said inner surface to said outer surface,
said first and second pivot ports shaped, positioned and dimensioned to engage said first and second pivot mounts whereby said ring is pivotally mounted to said yoke;
a multiplicity of corneal marking tabs,
each said corneal marking tab attached to said ring at said outer surface and extending outward therefrom,
each said corneal marking tab having a tab marker extending below said ring lower surface;
first and second axis marking tabs,
said first and second axis marking tabs attached to said ring inner surface and positioned to be diametrically opposed to each other,
whereby said ring can be rotated in said yoke to bring said corneal marking tabs or said axis marking tabs into contact with said cornea.

2. The apparatus as recited in claim 1 wherein said instrument has four said corneal marking tabs.

3. The apparatus as recited in claim 1 wherein said yoke is substantially semicircular in shape.

4. The apparatus as recited in claim 1 wherein at least two said corneal marking tabs are positioned go be diametrically opposite one another and one said axis marking tab is positioned opposite each said at least two corneal marking tabs.

5. The apparatus as recited in claim 1 wherein said yoke is dimensioned to allow said marking ring to rotate at least 360°.

6. The apparatus as recited in claim 1 wherein said marking ring has four said corneal marking tabs and two said axis marking tabs,
said corneal marking tabs positioned equidistantly around said marking ring.

7. A method for aligning an intraocular lens to replace a patient's diseased lens along said patient's previously-measured corneal steep axis by using an instrument to mark said patient's cornea, said corneal marking instrument having a marking ring pivotally mounted to an instrument yoke with said ring having opposed upper and lower surfaces with said upper surface having corneal marking tabs mounted thereon and said lower surface having axis marking tabs mounted thereon, said method comprising the steps of:
(a) rotating said marking ring to allow said corneal marking tabs to touch said cornea;
(b) coating said corneal marking tabs with an acceptable marking dye;
(c) contacting said cornea with said corneal marking tabs to make a first set of reference marks;
(d) placing a keratometer on said cornea and aligning said keratometer with said first set of reference marks;
(e) determining a reading on said keratometer to coincide with said corneal steep axis;
(f) rotating said marking ring to allow said axis mounting tabs to touch said cornea;
(g) coating said axis mounting tabs with an acceptable marking dye;
(h) aligning said axis mounting tabs with said keratometer such that said axis mounting tabs are aligned with said reading;
(i) contacting said cornea with said axis marking tabs to make a second set of reference marks;
(j) removing said diseased lens from the patient;
(k) inserting a replacement intraocular lens in place of the removed diseased lens to bring a selected portion of said lens into alignment with said second set of reference marks and, thereby, said corneal steep axis.

8. A method for verifying the alignment of an intraocular lens inserted to replace a patient's diseased lens along said patient's previously-measured corneal steep axis by using an instrument to mark said patient's cornea, said corneal marking instrument having a marking ring pivotally mounted to an instrument yoke with said ring having opposed upper and lower surfaces with said upper surface having corneal marking tabs mounted thereon and said lower surface having axis marking tabs mounted thereon, said method comprising the steps of:
(a) rotating said marking ring to allow said corneal marking tabs to touch said cornea;
(b) coating said corneal marking tabs with an acceptable marking dye;
(c) contacting said cornea with said corneal marking tabs to make a first set of reference marks;
(d) placing a keratometer on said cornea and aligning said keratometer with said first set of reference marks;
(e) determining a reading on said keratometer to coincide with said corneal steep axis;
(f) rotating said marking ring to allow said axis mounting tabs to touch said cornea;
(g) coating said axis mounting tabs with an acceptable marking dye;
(h) aligning said axis mounting tabs with said keratometer such that said axis mounting tabs are aligned with said reading;
(i) contacting said cornea with said axis marking tabs to make a second set of reference marks;
(j) removing said diseased lens from the patient;
(k) inserting said replacement intraocular lens in place of the removed diseased lens; and
(l) verifying that said replacement lens is in alignment with said corneal steep axis by comparing the position of said lens with said second set of reference marks.
